(19) United States
(12) Patent Application Publication
(10) Pub. No.: US 2024/0206763 A1
(43) Pub. Date: Jun. 27, 2024

(54) BIOLOGICAL SAMPLE ANALYSIS DEVICE WITH ASSOCIATED CAPTURING DEVICE AND ANALYSIS SOFTWARE

(71) Applicant: Agscent Pty Ltd, Carwoola, New South Wales (AU)

(72) Inventors: Bronwyn DARLINGTON, New South Wales (AU); Jessica DARLINGTON-BROWN, New South Wales (AU)

(21) Appl. No.: 18/574,018

(22) PCT Filed: Sep. 20, 2021
(86) PCT No.: PCT/AU2021/051083

§ 371 Date: Dec. 23, 2023

(30) Foreign Application Priority Data
Sep. 18, 2020 (AU) 2020903361

(51) Int. Cl. A61B 5/083; A61B 5/097

(52) U.S. Cl. CPC A61B 5/0836; A61B 5/097

(57) ABSTRACT

In one embodiment, there is described a biological sample analysis device. The device comprises a body including an outlet connectable to a chamber arranged to sealingly hold a biological sample and an inlet connectable to a mask portion arranged to fit over the nostrils of an animal to capture a breath sample from the animal. In a specific embodiment, there is provided an electrically operated valve located within the body and positioned between the inlet and outlet, and there is also provided a sensor located in the body and disposed at or near the inlet in a manner such that the sensor is capable of measuring the presence of at least one compound contained in the breath sample to provide an electrical signal indicative of the presence of the at least one compound to a microcontroller. In one embodiment, the microcontroller is arranged, upon determining the relative concentration of the at least one compound in the breath sample, and if the relative concentration is a desired concentration, the microcontroller moves the valve to an open condition, to allow the breath sample to flow into the chamber.

### TECHNICAL FIELD

The present technology relates to a biological sample analysis device. In some embodiments, the analysis device is associated with a capturing device and includes analysis software. In one embodiment, the invention relates to a device capable of capturing a breath sample from an animal, such as a cow, pig or sheep.

### BACKGROUND

It is known that biological samples from animals can yield a vast amount of information about the animal. For example, the blood of an animal carries certain chemical compounds and other substances (such as molecules or fragments of biological origin) which can be separated from the blood and analysed to determine the presence or absence of any one of a number of medical conditions, from disease and deficiency, through to specific biological conditions, such as pregnancy.

It is also known that some of the compounds found in bodily fluids such as blood are capable of crossing the lung barrier and traces of the compounds may be present in the exhaled breath of an animal. The presence and/or amount of the relevant compound or compounds present in the analysis of a chemical sample of an animal's breath can be used to determine the presence or absence of any one of a number of medical conditions. For example, one known technology which is applied to humans is the use of “breathalysers”, which are well known examples of chemical reaction based or electronic devices that include a chemical or electronic sensor that, when presented with a sample of a person's breath, detects the blood alcohol level of the person.

One of the advantages of using breath as a biological sample is that taking the sample is not invasive and is therefore less traumatic to the animal and potentially faster than a blood analysis.

However, the collection of a breath sample from an animal is not an easy task. Unlike a human, it is difficult or impossible to provide any meaningful instruction to an animal. For example, it is not possible to instruct an animal to breath out on command. Moreover, when faced with an unusual or unknown situation, such as having a device placed over the face, mouth or nostrils, the animal may act unusually, such as by pulling away, struggling, holding their breath, etc., which may result in the breath sample being contaminated by ambient air or not being of a sufficient volume to allow for accurate analysis.

It is with at least some of these issues in mind that the present invention has been developed.

In the context of the specification, the term “breath sample” encompasses any a captured mixture of gas and liquid exhaled from the lungs of an animal.

In the proceeding description, the acronym VOC refers to Volatile Organic Compounds (as found in the exhaled breath of an animal) and the term CO₂ refers to the compound carbon dioxide.

### SUMMARY OF THE TECHNOLOGY

In a first aspect, there is provided a biological sample analysis device comprising a body including an outlet connectable to a chamber arranged to sealingly hold a biological sample and an inlet connectable to a mask portion arranged to fit over the nostrils of an animal to capture a breath sample from the animal, an electrically operated valve located within the body and positioned between the inlet and outlet, and a sensor located in the body and disposed at or near the inlet in a manner such that the sensor is capable of measuring the presence of at least one compound contained in the breath sample to provide an electrical signal indicative of the presence of the at least one compound to a microcontroller, wherein the microcontroller is arranged, upon determining the relative concentration of the at least one compound in the breath sample, and if the relative concentration is a desired concentration, the microcontroller moves the valve to an open condition, to allow the breath sample to flow into the chamber.

In one embodiment, the device further comprises a temperature sensor arranged to provide an electric signal indicative of the temperature to the microcontroller, wherein the temperature signal is utilised by the microcontroller as an input value in the calculation of the relative concentration of the at least one compound.

In one embodiment, the device further comprises a secondary outlet, wherein the secondary outlet is in communication with the inlet and is openable by movement of the electrically operated valve, where if the relative concentration of the at least one compound is not a desired concentration, the microcontroller causes the valve to a closed condition, to prevent the breath sample from entering the chamber and consequently causes the breath sample to be ventilated through the secondary outlet.

In one embodiment, the device further comprises a one-way valve positioned intermediate the electrically operated valve and the outlet, wherein breath which flow into the chamber is prevented from exiting the chamber via the outlet.

The desired concentration of the at least one compound may be a predetermined range.

The at least one compound may be carbon dioxide.

The device may further include a flexible bag arranged to locate within the chamber, the bag being arranged sealingly over the outlet to capture the biological sample.

The chamber may be arranged to receive a total volume in the arrange of approximately 800 ml to 1000 ml and the flexible bag is arranged to receive a total volume in the range of approximately 800 ml to 1000 ml of breath.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is an exploded perspective view of a biological sample analysis device with a mask portion and associated capturing device in accordance with an embodiment of the invention;

FIG. 2 is a perspective view of a biological sample analysis device with a mask portion and associated capturing device in accordance with an embodiment of the invention;

FIGS. 3 and 4 are cross-sections of a diverter mechanism that forms part of the biological sample analysis device and associated capturing device, illustrating the diverter mechanism in open and closed conditions respectively;

FIG. 5 is a perspective view of a second embodiment of a biological sample analysis device with a mask portion and associated capturing device in accordance with an embodiment of the invention; and

FIG. 6 is an exploded perspective view of the second embodiment of the biological sample analysis device with a mask portion and associated capturing device in accordance with an embodiment of the invention.

### DETAILED DESCRIPTION OF EMBODIMENTS

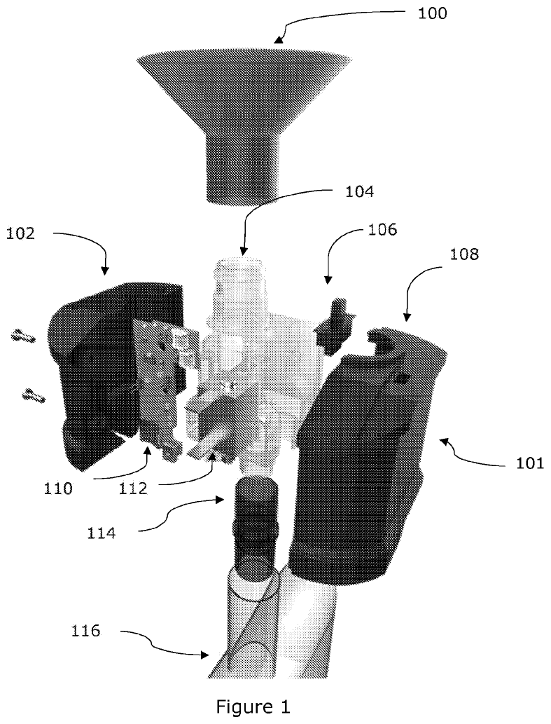

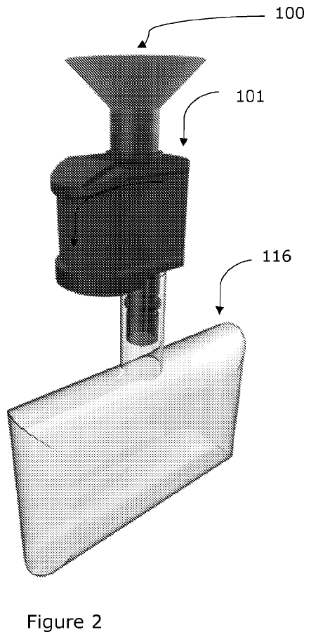

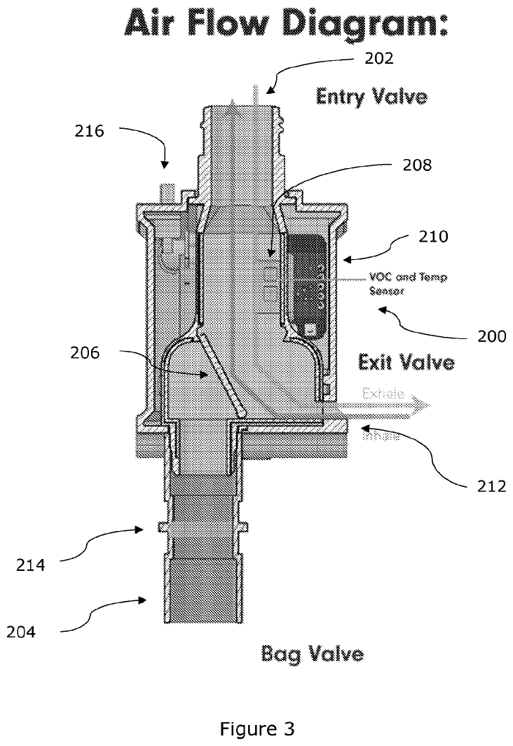

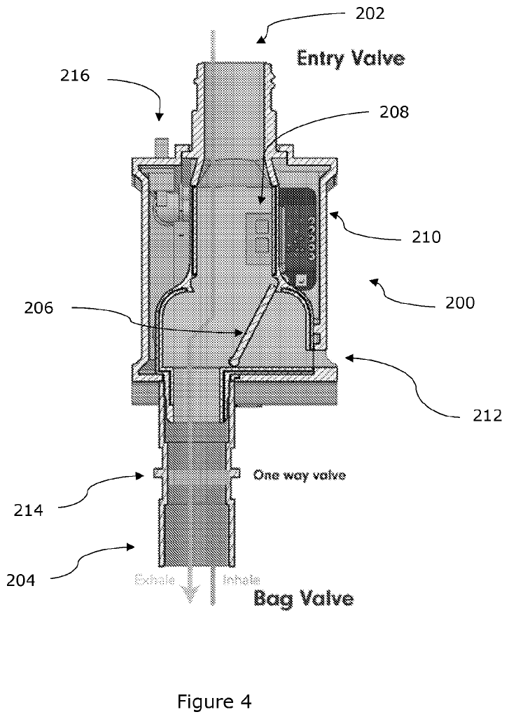

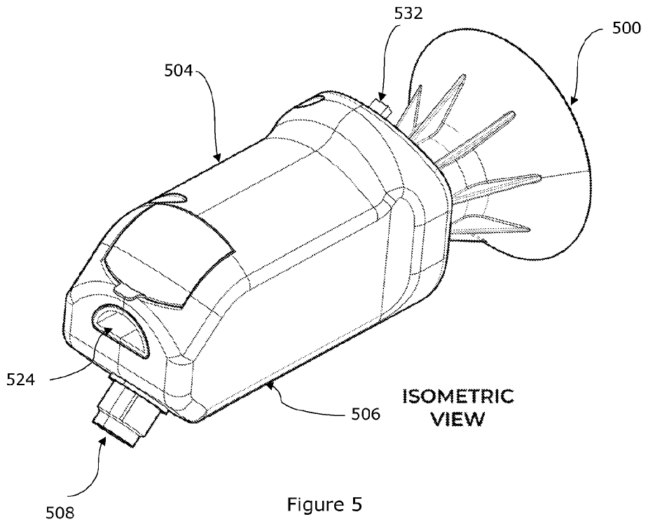

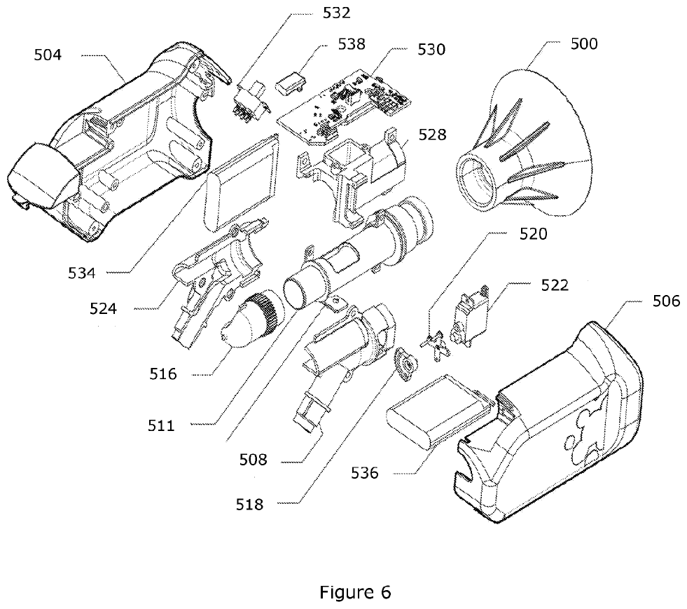

In the following detailed description of FIGS. 1 to 2, like numerals across FIGS. 1 and 2 refer to like features and/or integers. Similarly, in FIGS. 3 and 4, like numerals across FIGS. 3 and 4 refer to like features and/or integers. Lastly, in FIGS. 5 and 6, like numerals across FIGS. 5 and 6 refer to like features and/or integers.

Referring to FIGS. 1 through 2, there are shown various views of a device capable of capturing biological samples with an associated monitoring device. In one embodiment, the device is a breath capturing device capable of capturing the breath of an animal, for later analysis.

### Overview of Device (Referring to FIGS. 1 and 2)

At FIGS. 1 and 2 there is shown a device capable of capturing biological samples and analysing the samples in order to determine whether the sample should be captured or expelled into the atmosphere. In the embodiment shown, the biological sample is the breath of an animal. It will be understood that the device may be utilised to capture any type of animal breath, although in the example shown, the device is adapted to be particularly suited to capture the breath of a large farm animal, such as a cow, as the mask portion 100 (also termed a “snout” or a “nose cone”) is arranged to provide a good fit over the nostril of an animal such as a cow.

The device includes a sensor arrangement generally denoted by numeral 101 and also a collection receptacle 116, which may be lined with a flexible bag arranged to capture the biological sample (flexible bag not shown).

The sensor arrangement is housed in an outer shell comprised of two shell pieces 102 and 108 arranged to fit together. The sensor arrangement has an inlet 104 arranged to connect to the mask portion 100 and an outlet 114 which includes a one way valve arranged to direct the sample into the chamber 116.

The sensor arrangement includes a sensor 112 (described in more detail below) and a microcontroller 110 (also described in more detail below). There is provided a control switch 106.

### VOC Sensor Arrangement Embodiment

The sensor arrangement embodiment described herein functions as a measuring device which is arranged to be retrofitted onto a mask portion previously developed by the applicant and specifically designed to capture breath samples from larger animals such as cows, pigs and sheep. It will be understood, however, that the arrangement described herein may also be utilised, with minor variation in design, for any type of breath capture, including humans. Such variations are within the purview of a person skilled in the art.

In more detail, the arrangement also incorporates a two (2) way valve to allow the animal to breathe in and out normally while autonomously capturing the breath of the animal and preventing the captured air from escaping either due to pressure differences or when the animal breathes in.

The electronic sensor is integrated with a microcontroller (Arduino). The microcontroller is specifically selected and/or tuned to detect a wide range of VOCs and an equivalent of CO₂ (eCO₂), the sensor producing a signal which is utilised by the microcontroller to determine the concentration levels on the cows' breath.

In the embodiment, data is transferred bit by bit along a single wire from the sensor to the microcontroller using the I2C communication protocol. There is also provided, in the embodiment, an onboard thermistor used to calculate the local ambient temperature, which is also communicated to the microcontroller and utilised as an input to determine the relative concentration of VOCs and eCO₂.

In the embodiment described herein, the sensor and microcontroller are capable of detecting concentration ranges of 400 to 8192 parts per million (ppm) CO₂, and TVOC (Total Volatile Organic Compound) concentration within a range of 0 to 1187 parts per billion (ppb)

The Applicant has found that CO₂ levels are related to the relative length of breath, and that higher CO₂ levels are consistent with the capture of a higher level of VOCs, which in turn allow for a high quality result when the captured breath is analysed for the presence of specific biomarkers (such as biomarkers that indicate pregnancy of the animal).

It will be understood that the “desired” level of CO₂ concentration is determined by a number of factors, including the animal type, other environmental conditions, the relative sensitivity of the sensor and the analysis technique employed to determine the presence of biomarkers. Therefore, the relative amounts and concentrations provided in the present specification apply to the particular sensors and conditions described with reference to the example embodiment described herein, and it will be understood that a person skilled in the art would calibrate a given sensor against known sources in order to provide optimal results. Such calibration is a known technique to a person skilled in the art and falls within the purview of a person skilled in the art.

### Example of Desired Concentration of CO₂ for Optimal Biomarker Collection

Upon performing various experiments on a specific animal type, namely cows, the applicant has determined that approximately 800 ml-1000 ml of cow breath provides a richer source of VOCs when compared with a 500 ml sample. Moreover, while 2000 ml also provides a rich source, 1000 ml is a more practical amount to capture.

Using 800 ml of cow breath the applicant was capable of identifying six (6) VOCs which clearly separate pregnant and non-pregnant cattle. A separation was also seen in the 500 ml breath sample, however the distinction between pregnant and non-pregnant cattle was greatly improved when using 800mi-1000 ml. In the embodiment described herein, the collection cannister/bag is arranged to hold approximately 800 ml-1000 ml of breath. However, it will be understood that variations of the device with larger collection volumes are within the purview of a person skilled in the art, and 1000 ml has been found to be a suitable amount for the intended analysis given in the present specification.

Moreover, the applicant has found that the use of particular plastics that are known for having very low “off-gassing” rates also operate to decrease the possibility of contaminants entering the sample of captured breath. For example, using a sample collection bag material made from Polytetrafluoroethylene (PTFE) improved clarity in the results and the embodiment described herein utilises sample collection bags made from a PTFE material. However, it will be understood that any material with similar low off gassing properties may be utilised.

The sensor embodiment described herein was tested on twenty (20) pregnant cows at Carwoola Pastoral company. Two readings were taken from each cow. The first reading was a CO₂ only assessment. The cow was secured in the crush with its head in the chin lift. The mask portion and CO₂ sensor was placed over each cow's nostril and held for up to 60 seconds (approximately 2-3 full breath cycles) to collect a range of CO₂ data. The CO₂ data was immediately visually available using Adino software and recorded directly to a memory disk, and the range of CO₂ was noted.

For the second sample, a bag made from a Nalophan material was attached to a one-way valve and secured to the CO2 sensor and the snout was placed over the cow's nostril. Nalophan is a trade mark name for a material that is suitable for the construction of flexible bags useful for the collection of biological samples such as breath, as Nalophan also has low offgassing properties when breath samples are only held in the bag for relatively short periods of time). A breath sample was collected from each cow and the CO₂ range was also noted. A total of 10 one litre breath samples were collected and two litre breath samples were collected (from different cows).

The data is outlined below in Table 1:

| Cow | | Litres of breath | Range from CO₂ | Upper |
|---|---|---|---|---|
| ID | CO2 | collected | sensor | range |
| 00060 | 0.9 | 2 | 2000-9000 | 9000 |
| 00072 | 0.7 | 1 | 800-2400 | 2400 |
| 00074 | 1.1 | 1 | 3000-4000 | 4000 |
| 00099 | 2.4 | 1 | 800-1600 | 1600 |
| 00230 | 2.3 | 2 | 600-2400 | 2400 |
| 00985 | 1.8 | 2 | 1500-4500 | 4500 |
| 01004 | 1.5 | 2 | 2500-7500 | 7500 |
| 01043 | 0.4 | 2 | 400-1200 | 1200 |
| 02847 | 1.2 | 1 | 4000-7500 | 7500 |
| 02894 | 1.3 | 2 | 1500-6000 | 6000 |
| 03009 | 0.3 | 2 | 1500-4700 | 4700 |
| 03257 | 1.8 | 1 | 1500-4500 | 4500 |
| 04587 | 0.9 | 2 | 600-2000 | 2000 |
| 04600 | 1.3 | 1 | 600-2000 | 2000 |
| 04609 | 1.2 | 1 | 600-2400 | 2400 |
| 04625 | 1.4 | 2 | 4000-13000 | 13000 |
| 04626 | 1 | 1 | 1500-4500 | 4500 |
| 04704 | 0.5 | 2 | 2000-6000 | 6000 |
| 04717 | 0.5 | 1 | 1000-4000 | 4000 |
| No Tag | 1.7 | 1 | 1500-5000 | 5000 |

The results show that each cow has varying levels of CO₂ in their breath. The lower limit range was between 600-300 and the upper limit varied between 1200-13000.

The theory for using CO₂ as an indicator of rich VOC is based on the biological rationale that high levels of CO₂ is from alveolar breath which is air from the deepest part of the lungs. The applicant has progressed on the assumption that higher CO₂ and VOC correlate. In other words, as CO₂ increases, so does the richness of VOC in the breath sample. Therefore, based on experimental evidence produced under the conditions described above, it was found that there is a boundary effect that eliminates the lowest 20% of CO2 values. This provides a lower limit which is utilised to calibrate the sensor and determine when the diverter (described below) operates.

Moreover, an upper limit is also determined as it is hypothesised that very high CO₂ values may be due to CO₂ produced from the gut of the animal, not from breath as such. Therefore, the device is arranged to also exclude breath with very high CO₂ levels (i.e. the diverter system excludes breaths with very high CO₂ levels).

### Diverter (Valve) System—Overview

FIGS. 3 and 4 provide a more detailed view of the diverter system 200, which incorporates the sensor system (208 and 210), in accordance with an embodiment of the invention.

The diverter system 200 has an inlet 202 and an outlet 204, and an electric valve 206 which is controlled (as described below) by the microcontroller 210. The microcontroller 210 is provided with sensor data from sensor 208.

The valve is arranged to move to allow a captured breath sample to be either diverted to outlet 204 if the breath sample is to be captured, or to outlet 212 if the breaths ample is to vented to the outside atmosphere.

To prevent a captured sample from “leaking” into the diverter system, a one way valve 214 is locate between the diverter system and the outlet 204.

### Diverter (Valve) System—Operation

The diverter (valve) system comprises an open-close valve system to divert concentrated breath samples from the animal to either the collector or to the external atmosphere dependent on the quality of the sample. The device incorporates an SGP30 sensor (capable of capturing and sending data indicative of the amount of VOCs and exhaled CO₂ in the breath sample, in real time) that directs the air to a PTFE Bag once the optimal VOC and exhaled CO₂ levels are reached. The samples in the bag are then taken to another device (not shown) for an accurate measurement of VOCs for the purpose of the detection of pregnancy of the animal.

In one embodiment, the cannister is designed in an ergonomic fashion to allow the device to be utilised using a one-handed grip, to allow the user to have a free hand for other reasons, such as steadying or comforting the animal.

The Diverter system includes a microcontroller programmed to receive the data from the sensor and analyse the data, and in one embodiment, also captures the data from the sensor onto an electronic storage device, which in the embodiment shown, is a “SD” memory card device.

It will be understood that the device also includes other components required for the operation of the aforementioned components, including a power source such as a rechargeable battery. It will be further understood that the device will also include other known components, such as a means for charging the battery, a power switch, a reset button, and in some embodiments, a visual or audible indicator means, such as a screen or a speaker, such that the device can provide feedback to the user and correspondingly a user can operate the device. Such features form part of the embodiment of the invention.

The Internal diverter valve and flap in the embodiment shown are manufactured from a Talmans (brand name) t-glase PETT plastic, which is a commercially available plastic arranged to produce a very low level of “off gassing”. Similarly, the surrounding housing of the embodiment is manufactured from Onyx (brand name) nylon blend with carbon fibre, again to reduce off gassing and to provide a robust mechanism that is more likely to survive and continue to operate in a harsh environment. It will be understood that any suitable materials may be utilised to construct the diverter system.

### Utilising the Automated Device

When the device is placed against the nostril of a large animal, such as a cow, the nose piece is placed over the nose of the animal, and when the animal breathes out into the nose piece, air is directed to the outside of the diverter, which acts as an exit valve for air that does not meet the CO₂ requirements for rich sample data, while allowing the animal to breathe in and purge the CO2 sensor.

Subsequently, when the VOC sensor measures an optimum range of CO₂ in the sampled breath; the solenoid pulls the actuator flap over, redirecting the flow of air past the one way valve and into the sample bag. When the CO₂ level drops the solenoid releases and a band helps to pull the actuator flap down, snapping the flap back to the starting position. The one way valve stops the captured breath in the bag/cannister from being inhaled once collected in the bag.

### Second Embodiment of Breath Capture Device

Referring to FIGS. 5 through 6, there is shown a perspective and an exploded view of a second embodiment of a device capable of capturing biological samples with an associated monitoring device. In the second embodiment, the device is, like the previous embodiment, a breath capturing device capable of capturing the breath of an animal, for later analysis.

### Overview of Device (Referring to FIGS. 5 and 6)

At FIGS. 5 and 6 there is shown a second embodiment of a device capable of capturing biological samples and analysing the samples in order to determine whether the sample should be captured or discarded (expelled into the atmosphere). In the second embodiment shown, the biological sample is the breath of an animal. It will be understood that the device of the second embodiment may be utilised to capture any type of animal breath, although in the example shown, the device is adapted to be particularly suited to capture the breath of a large farm animal, such as a cow, as the mask portion 500 (also termed a “snout”, a “nose cone” or a “bung”) is arranged to provide a good fit over either one or both of the nostrils of an animal such as a cow.

The device 500 includes a body (shell) generally denoted by numeral 502 comprised of two shell portions 504 and 506 arranged to fit together, and once fitted, the shell portions, amongst other features, define an outlet 508, onto which may be affixed a collection container and/or a flexible bag arranged to capture the biological sample (collection container and/or flexible bag not shown).

The sensor arrangement is comprised of a number of components, housed in the shell 502. In the context of the second embodiment, the term “sensor arrangement” is utilised as a “shorthand” to describe the physical connection and/or interworking relationship between a number of components that comprise the embodiment. However, it will be understood that the term “sensor arrangement” is utilised in the following detailed description in order to provide context for the embodiment as a whole and the term should not be construed as being limited to, or conversely, including all of, the integers or features described herein. It will be understood that the invention defined herein is limited to the features explicitly defined in the claims, and no gloss should be drawn from the term “sensor arrangement” as described in the proceeding detailed description, in order to imply the presence or absence of features from the claimed invention.

The sensor arrangement includes an inlet port 510 arranged to connect to a diverter chamber 511 (comprised of shell portions 512 and 514 in FIG. 6), the diverter chamber having an outlet that is connected to outlet 508.

The diverter chamber 511 incorporates a gate 516 which functions as a one-way valve, the gate 516 being connected to a gear 518 and correspondingly, the gear is connected via a gear mount 520 to a servo motor 522. The servo motor 522 functions to turn the gear 518 to move the gate 516, to thereby divert a breath sample to one of two outlets (i.e. the outlet 508 or the exhaust outlet 524).

The sensor arrangement also includes a sensor 526 arranged to be housed in the sensor mount 528, whereby the sensor 526 is exposed to an inside surface of the inlet port 510 via window 528. The sensor 526 is in communication with a microcontroller (and associated circuitry) 530. The microcontroller 530 is arranged to receive data from the sensor 526 to control the servo motor 522. The microcontroller 530 is controlled, in part, by switch 532.

Two batteries 534 and 536 provide power to the motor 522, the sensor 526 and the microcontroller 530. There is also provided a light 538, which provides feedback to the user on various functions.

In operation, the sensor arrangement operates in the same functional manner as the first embodiment.

### Advantages

One of the advantages of the embodiments and broader invention described herein is that the device provides a cost effective, reliable and reusable device for capturing a breath sample to a required standard by determining the presence and/or concentration of a specific compound, to deduce whether the breath sample is adequate for the purpose of more detailed analysis of the volatile organic compounds contained in the breath sample.

Moreover, in one embodiment, the automation of the diverter allows a user to easily collect a breath sample that is of a desired standard or quality from an animal without requiring any specific training, understanding of the underlying working principles of the device, or requiring the user to have any specialist knowledge of any “rules” or specific procedures for determining the standard or quality of the breath sample.

### Definition and Clarification of Terms

Throughout this specification, unless the context requires otherwise, the word “comprise” or variations such as “comprises” or “comprising”, will be understood to imply the inclusion of a stated integer or group of integers but not the explicit exclusion of any other integer or group of integers.

Those skilled in the art will appreciate that the embodiments described herein are susceptible to obvious variations and modifications other than those specifically described and it is intended that the broadest claims cover all such variations and modifications. Those skilled in the art will also understand that the inventive concept that underpins the broadest claims may include any number of the steps, features, and concepts referred to or indicated in the specification, either individually or collectively, and any and all combinations of any two or more of the steps or features may constitute an invention.

Where definitions for selected terms used herein are found within the detailed description of the invention, it is intended that such definitions apply to the claimed invention. However, if not explicitly defined, all scientific and technical terms used herein have the same meaning as commonly understood to one of ordinary skill in the art to which the invention belongs.

1. A biological sample analysis device comprising a body including an outlet connectable to a chamber arranged to sealingly hold a biological sample and an inlet connectable to a mask portion arranged to fit over the nostrils of an animal to capture a breath sample from the animal, an electrically operated valve located within the body and positioned between the inlet and outlet, and a sensor located in the body and disposed at or near the inlet in a manner such that the sensor is capable of measuring the presence of at least one compound contained in the breath sample to provide an electrical signal indicative of the presence of the at least one compound to a microcontroller, wherein the microcontroller is arranged, upon determining the relative concentration of the at least one compound in the breath sample, and if the relative concentration is a desired concentration, the microcontroller moves the valve to an open condition, to allow the breath sample to flow into the chamber.

2. The device according to claim 1, further comprising a temperature sensor arranged to provide an electric signal indicative of the temperature to the microcontroller, wherein the temperature signal is utilised by the microcontroller as an input value in the calculation of the relative concentration of the at least one compound.

3. The device according to claim 1, further comprising a secondary outlet, wherein the secondary outlet is in communication with the inlet and is openable by movement of the electrically operated valve, where if the relative concentration of the at least one compound is not a desired concentration, the microcontroller causes the valve to a closed condition, to prevent the breath sample from entering the chamber and consequently causes the breath sample to be ventilated through the secondary outlet.

4. The device according to claim 1, further comprising a one-way valve positioned intermediate the electrically operated valve and the outlet, wherein breath which flow into the chamber is prevented from exiting the chamber via the outlet.

5. The device according to claim 1, wherein the desired concentration of the at least one compound is a predetermined range.

6. The device according to claim 1, wherein the compound is carbon dioxide.

7. The device according to claim 1, further including a flexible bag arranged to locate within the chamber, the bag being arranged sealingly over the outlet to capture the biological sample.

8. (canceled)

9. The device according to claim 7, wherein the flexible bag is arranged to receive a total volume in the range of approximately 800 ml to 1000 ml of breath.

10. The device according to claim 1, wherein the chamber is arranged to receive a total volume in the range of approximately 800 ml to 1000 ml.
